(19) United States
(12) Patent Application Publication
(10) Pub. No.: US 2005/0197591 A1
(43) Pub. Date: Sep. 8, 2005

(54) System and method for prebalancing electrical properties to diagnose disease

(75) Inventors: Zoran Pavlovic, Toronto (CA); Milan Graovac, Toronto (CA); Joel Steven Ironstone, Toronto (CA)

Correspondence Address: BERESKIN AND PARR, 40 KING STREET WEST, BOX 401, TORONTO, ON, M5H 3Y2, CA

(73) Assignee: Z-Tech (Canada) Inc., Toronto (CA)

(21) Appl. No.: 10/790,846

(22) Filed: Mar. 3, 2004

(51) Int. Cl. A61B 5/05; A61B 5/00

(52) U.S. Cl. 600/547

(57) ABSTRACT

A system and method for diagnosing the possibility of disease by making electrical measurements in one of a first body part and a second substantially similar body part are described. The present invention balances out differences between homologous body parts that are due to natural factors unrelated to disease, such as differences in size or symmetry between left and right breasts. Once data are prebalanced, statistical analyses can be performed on the data to diagnose disease. The system includes a normalizing module for obtaining a normalizing factors database from a screening population group to account for differences in spatial separation of impedance measurements. Once a set of normalizing factors is obtained, a prebalancing factor can be obtained that can further be used to adjust raw electrical measurements. Normalizing factors are applied to a smaller subset of measurements that are likely to better represent the body part as a whole. This set of measurements is reduced further by eliminating a set of the measurements that can be biased by a presence of a disease in a body part. The remaining measurements for each body part are then averaged to obtain an overall measure of a body part electrical property. The quotient between these measures is then used to adjust raw measurements. The adjusted measurements remove the imbalance that might exist due to natural differences between body parts. Adjusted measurements are then used as an input to other methods to obtain more accurate disease diagnostics.

### FIELD OF THE INVENTION

This invention relates to a method for detecting and diagnosing disease states in living organisms and specifically relates to diagnosis of disease by measuring electrical properties of body parts.

### BACKGROUND OF THE INVENTION

Several methods exist for diagnosing disease that involve measuring a physical property of a part of the body. A change in such a physical property can signal the presence of disease. For example, x-ray techniques measure tissue physical density, ultrasound measures acoustic density, and thermal sensing techniques measures differences in tissue heat generation and conduction. Other properties are electrical, such as the impedance of a body part that is related to the resistance that the body part offers to the flow of electrical current through it.

Values of electrical impedance of various body tissues are well known through studies on intact humans or from excised tissue made available following therapeutic surgical procedures. In addition, it is well documented that a decrease in electrical impedance occurs in tissue as it undergoes cancerous changes. This finding is consistent over many animal species and tissue types, including, for example human breast cancers.

A method for using electrical properties to diagnose disease involves homologous body parts, i.e., body parts that are substantially similar, such as a left breast and a right breast. In this method, the impedance of a body part of a patient is compared to the impedance of the homologous body part of the same patient. One technique for screening and diagnosing diseased states within the body using electrical impedance is disclosed in U.S. Pat. No. 6,122,544, which is incorporated herein by reference. In this patent, data are obtained from two anatomically homologous body regions, one of which may be affected by disease. Differences in the electrical properties of the two homologous body parts could signal disease.

To draw such a conclusion, it is assumed that, in the absence of disease, the two homologous body parts are sufficiently similar, and, ideally, identical. However, the difference may also arise because of natural variability between body parts, such as variability due to size or structural differences, or the effect of different surrounding tissues. If measured impedances are used directly, the natural variability can skew the results and a faulty diagnosis may result, such as showing disease in a body part.

### SUMMARY OF THE INVENTION

The present invention balances out differences between homologous body parts that are due to natural factors unrelated to disease, such as differences in size or symmetry between left and right breasts. Once data are prebalanced, statistical analyses can be performed on the data to diagnose disease.

In particular, a method for diagnosing the possibility of disease in one of a first body part and a second substantially similar body part is described herein. The system includes a normalizing module for obtaining a normalizing factors database from a screening population group to account for differences in spatial separation of impedance measurements. This module normalizes a set of measurements within a body part. Once a set of normalizing factors is obtained, a prebalancing factor can be obtained that can further be used to adjust raw electrical measurements. Normalizing factors are applied to a smaller subset of measurements that are likely to better represent the body part as a whole. This set of measurements is reduced further by eliminating a set of the measurements that can be biased by a presence of a disease in a body part. The remaining measurements for each body part are then averaged to obtain an overall measure of a body part electrical property. The quotient between these measures is then used to adjust raw measurements. The adjusted measurements remove the imbalance that might exist due to natural differences between body parts. Adjusted measurements are then used as an input to other methods, such as HEDA (PCT/CA01/01788) to obtain more accurate disease diagnostics.

More particularly, a method and system for diagnosing the possibility of disease in one of a first body part and a second substantially similar body part is described herein. The system includes a prebalancing factor module for obtaining a prebalancing factor (PBF) from a population group to account for variability between the first body part and the second body part. The system also includes an electrode array for measuring a first electrical property of the first body part and a second electrical property of the second body part. The system further includes a prebalancing module for utilizing the prebalancing factor to prebalance at least one of the first electrical property and the second electrical property. The prebalanced first electrical property and second electrical property can be used to diagnose the possibility of disease in one of the first body part and the second body part.

### BRIEF DESCRIPTION OF THE DRAWINGS

FIG. 1 is a flow/system block diagram of the normalizing factor module of the diagnostic system;

FIG. 2 is a flow/system block diagram of the prebalancing factor module of the diagnostic system; and

FIG. 3 is a flowchart illustrating the method steps performed by the diagnostic system of FIG. 1 and FIG. 2 to diagnose disease in a body part.

### DETAILED DESCRIPTION OF THE INVENTION

Normalizing Factors Module

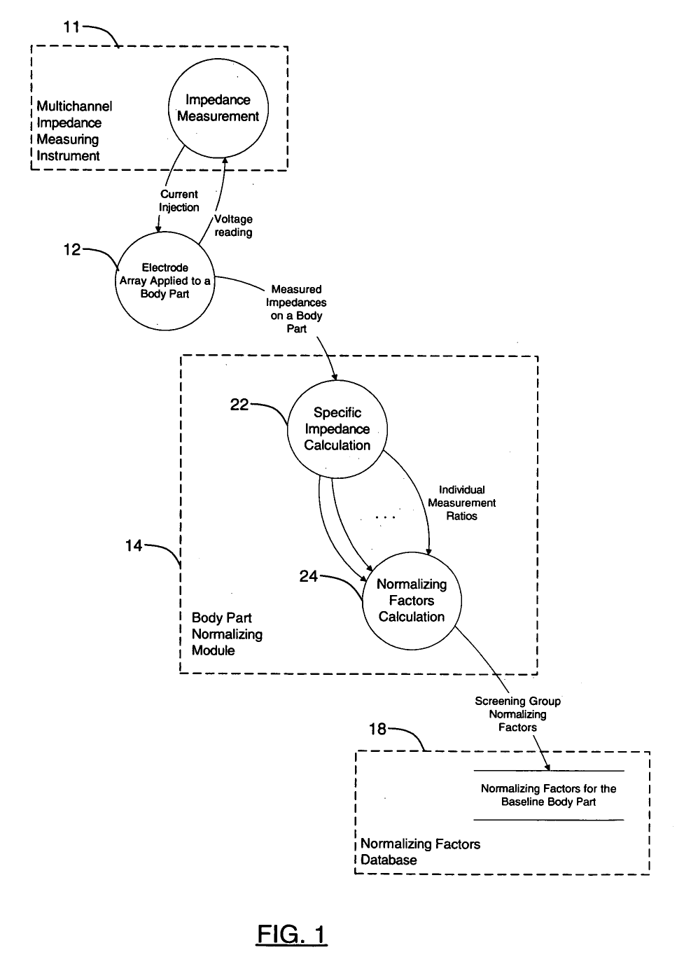

FIG. 1 shows a flow/system diagram for detecting and diagnosing disease, such as a breast cancer. The system of FIG. 1 includes a multi-channel impedance-measuring instrument 11, an electrode array 12, a normalizing module 14 and a normalizing factors database 18. In one embodiment, the electrode array 12 includes nₑ current injection electrodes, and nₑ voltage measurement electrodes. The electrodes are applied to the body part, and each of the current injection electrodes is associated with the adjacent voltage measurement electrode. Impedance is calculated by measuring the voltage between two voltage electrodes when the current is injected between the associated current electrodes. The total number of independent current injections and related impedances is n_{Cl}=nₑ·(nₑ−1)/2.

Normalization factors are calculated from a population of N_{g} subjects who have no disease in a body part of interest (e.g. women with disease-free breasts). For each subject, n_{Cl} impedance measurements,

$$\{ {Z}_{i , j}^{first K} \} ,$$

and n_{Cl} impedance measurements,

$$\{ {Z}_{i , j}^{sec K} \} ,$$

are acquired, where

$${Z}_{i , j}^{first K}$$

is the impedance of the first body part measured between voltage electrodes i and j when current is injected between associated current electrodes, for the K^{th} subject. For each measurement the specific impedance calculation module 22 calculates:

$${M}_{i , j}^{K} = \frac{{Z}_{i , j}^{K}}{{d}_{i , j}}$$

where M_{i,j}^{K} is a specific impedance (i.e., impedance per distance), Z_{i,j}^{K} is the measured impedance between voltage electrodes i and j, d_{i,j} is related to the distance between the electrodes. (In the last equation and in the rest of this section, the superscripts “first” and “sec” are omitted for clarity of notation; however, it should be understood that these are implied where quantities pertain to the first or second body part.). In one embodiment the Euclidean distance is measured between the voltage electrodes i and j on the electrode array 12 while the electrode array is placed on a realistic model of a body part. In other embodiments, a different metric can be employed that accounts for the curvature of the electrode array, which duplicates the curvature of the breast.

Further, a pair of electrodes (ref1, ref2) are selected, and its specific impedance designated as a reference measurement (M_{ref}). The reference measurement electrodes are the same over the entire subject population. The normalizing quotients for subject K can be calculated as:

$${q}_{i , j}^{K} = \frac{{M}_{i , j}^{K}}{{M}_{ref}}$$

for each pair of electrodes (i,j). The normalizing quotients differ based on the position of electrodes on the body part (e.g. on a breast there is a significant difference between measurements in the inner lower region, as compared to the outer upper region).

The normalizing factors calculation module 24 repeats the previous steps in all members of the population group to obtain the set of quotients, {q_{i,j}¹,q_{i,j}², . . . q_{i,j}^{Ng}}, the superscripts denoting the various members of the group.

The normalizing factor calculation module 24 calculates a set of normalizing factors r_{i,j} given by:

$${r}_{i , j} = \frac{1}{{N}_{g}} {\sum }_{K = 1}^{{N}_{g}} {q}_{i , j}^{K} .$$

The steps leading to the normalizing factors r_{i,j} are performed on a population group with no disease. These values may then be stored in the normalizing factors database 18.

Prebalancing Factor Module

The prebalancing factor module 16 includes software and/or hardware for obtaining a prebalancing factor PBF from a population group to account for variability between the first body part and the second body part, as described in more detail below. For example, if the first and the second body part are right and left breasts, variability can arise because of size or architectural differences. This variability can skew results when comparing the right and left breasts, and cause faulty diagnosis. The present invention attempts to eliminate such natural variability between the first and the second body part by prebalancing so that differences that do arise can be attributed more confidently to the presence of disease.

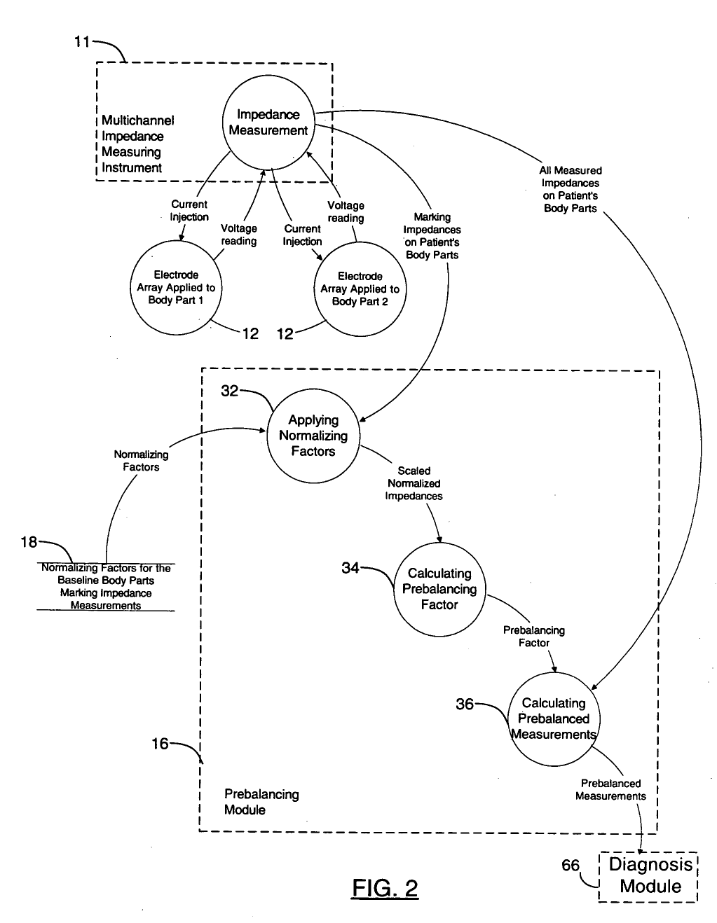

Referring to FIG. 2, the method uses impedance measurements taken from the multi-channel impedance measuring instrument 11 with the pair of electrode arrays 12 such as the one described in PCT/CA01/01788 which is incorporated herein by reference, plus the normalizing factors database 18, and prebalancing module 16.

The electrodes of the electrode array 12 are applied on the patient, the multi-channel impedance measurement instrument 11 measures electrical properties (e.g. impedances) of two substantially similar body parts, such as a left and a right breast.

A small subset of all measurements that characterize the body part is taken. In the case where the first and the second body part are human breasts, it is advantageous that 1) the distance between the electrode pairs in the subset is approximately the same; 2) the electrodes are disposed at the outer area of the breast, and 3) the separation between electrodes in the pairs embraces about a quarter of the breast circumference.

Normalizing factors obtained from a normalizing factors database 18 are applied to the subset of first and second body part measurements 32, as follows:

$${Znorm}_{i , j}^{first} = {Z}_{i , j}^{first} / {r}_{i , j}^{first} and {Znorm}_{i , j}^{sec} = {Z}_{i , j}^{sec} / {r}_{i , j}^{sec}$$

where

$${Z}_{i , j}^{first}$$

is the impedance measured between voltage electrodes i and j when current is injected between associated current injection electrodes i and j. In particular, the impedance may be obtained according to

$${V}_{i , j}^{first} / {I}_{i , j}^{first} ,$$

where

$${V}_{i , j}^{first}$$

is the voltage difference between electrodes i and j when a current

$${I}_{i , j}^{first}$$

is injected between associated current injection electrodes i and j.

This yields a normalized subset of impedances for both body parts. These subsets are pared down further to yield a final (and smaller) normalized subset by removing normalized impedances that could correspond to anomalous electrical pathways. For example, these subsets can be formed by removing approximately half of the smallest values of the normalized impedances. These smaller values are removed because they could potentially correspond to electrical pathways encountering malignant tumors. The highest value of the set, which could be an outlier, may also be removed. (Alternatively, more than one, e.g., the two highest values can be removed). The values in the final normalized subsets are averaged as follows:

$${Znorm}^{first} = \frac{1}{n} {\sum }_{p = 1}^{n} {Znorm}_{p}^{first} and {Znorm}^{sec} = \frac{1}{{n}^{′}} {\sum }_{p = 1}^{{n}^{′}} {Znorm}_{p}^{sec}$$

where each

$${Znorm}_{p}^{first}$$

is associated with a particular pair of electrodes, the sum running over the corresponding pairs that contribute to the subset. Thus, n≦n_{Cl} and n′≦n_{Cl}. The prebalancing factor PBF is then calculated in the prebalancing factor calculator module 34:

$$PBF = \frac{{Znorm}^{sec}}{{Znorm}^{first}} .$$

The prebalancing module 36 prebalances all impedance measurements

$${Z}_{i , j}^{first} and {Z}_{i , j}^{sec}$$

to yield

$${Z}_{i , j}^{{first}^{*}} and {Z}_{i , j}^{{sec}^{*}} ,$$

where

$${Z}_{i , j}^{{first}^{*}} = PBF \cdot {Z}_{i , j}^{first} and {Z}_{i , j}^{{sec}^{*}} = {Z}_{i , j}^{sec} ,$$

if PBF is greater than one, and

$${Z}_{i , j}^{{first}^{*}} = {Z}_{i , j}^{first} and {Z}_{i , j}^{{sec}^{*}} = {Z}_{i , j}^{sec} / PBF ,$$

if PBF is less than one.
Once the raw impedance measurements have been prebalanced, the prebalanced values can be processed to diagnose disease with a diagnosis module 66. For example, statistical tests can be performed to determine if significant differences exist between the right and left breast that could signal disease. Examples of such diagnostic procedures that can be performed are described in U.S. Pat. No. 6,122,544.

Different computer systems can be used to implement the method for diagnosing a disease in a body part. In one embodiment, the method can be implemented on a 2 GHz Pentium™ system with 512 Mb RAM.

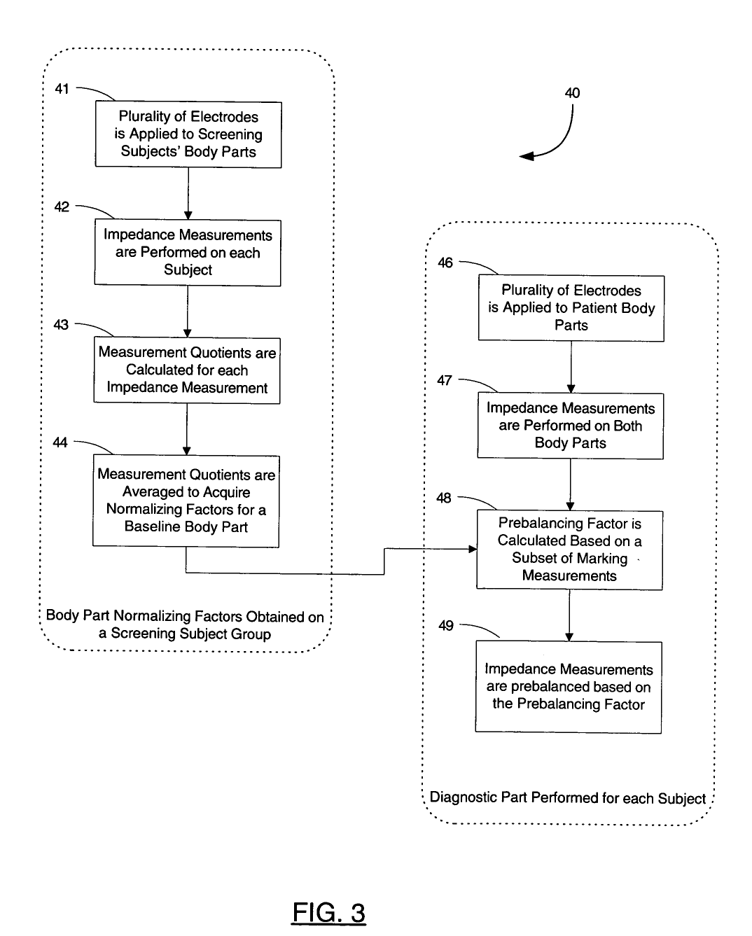

FIG. 3 shows a flowchart which illustrates the steps performed for diagnosing the possibility of disease in a body part. At the application step (41), a plurality of electrodes is applied to a set of screening subjects, and impedance measurements are performed on each subject (42). Next, a set of normalizing quotients is obtained for each subject (43). These quotients are averaged to obtain a database of normalizing factors (44). The above steps are performed only once to obtain the normalizing factors database.

For each subject to be diagnosed the following steps are performed. A plurality of electrodes is applied to both body parts (46) and impedance measurements are taken (47). A prebalancing factor is calculated based on a subset of measurements and normalized factors database (48). All impedance measurements are prebalanced using the calculated prebalancing factor (49).

It should be understood that various modifications and adaptations could be made to the embodiments described and illustrated herein, without departing from the present invention, the scope of which is defined in the appended claims. For example, although emphasis has been placed on describing a system for diagnosing breast cancer, the principles of the present invention can also be advantageously applied to other diseases of other body parts. In addition, the same principles of the present invention used to prebalance impedance measurements can be used to prebalance other electrical or non-electrical measurements, such as acoustic impedance measurements. Moreover, there are several reasons to prebalance electrical properties besides the diagnosis of disease. For example, electrical data can be prebalanced for the purpose of conducting research, to characterize normal electrical differences between homologous body parts. The method for prebalancing can be used as a predictor of homologous differences as measured by tissue physical density or acoustic transmission properties. A set of “normal or unaffected” values within a larger set may be sought that may contain members that are likely to be outside the normal set. The method and system described herein may then be used to prebalance the appropriate values.

1. A method for prebalancing an electrical property obtained from at least one of a first body part and a second substantially similar body part, the method comprising the steps of:
obtaining a prebalancing factor (PBF) from a population group to account for variability between the first body part and the second body part;
measuring an electrical property of at least one of the first body part and the second body part with an electrode array; and
utilizing the prebalancing factor to prebalance the electrical property.

2. The method of claim 1, wherein the first and second body parts are breasts.

3. The method of claim 1, wherein the electrode array includes a plurality of current injection electrodes and a plurality of voltage measurement electrodes.

4. The method of claim 3, wherein the electrical property is electrical impedance, and wherein the step of measuring includes
injecting currents into the first body part with the plurality of current injection electrodes;
measuring a set of impedances
$$\{ {Z}_{i , j}^{first} \}$$
with the plurality of voltage measurement electrodes;
injecting currents into the second body part with the plurality of current injection electrodes; and
measuring a set of impedances
$$\{ {Z}_{i , j}^{sec} \}$$
with the plurality of voltage measurement electrodes.

5. The method of claim 4, wherein the step of utilizing the prebalancing factor includes
prebalancing
$$\{ {Z}_{i , j}^{first} \} and \{ {Z}_{i , j}^{sec} \}$$
to yield the sets
$$\{ {Z}_{i , j}^{{first}^{*}} \} and \{ {Z}_{i , j}^{{sec}^{*}} \} ,$$
where
$${Z}_{i , j}^{{first}^{*}} = PBF \times {Z}_{i , j}^{first} and {Z}_{i , j}^{{sec}^{*}} = {Z}_{i , j}^{sec} , if PBF \geq 1 , and$$
$${Z}_{i , j}^{{first}^{*}} = {Z}_{i , j}^{first} and {Z}_{i , j}^{{sec}^{*}} = {Z}_{i , j}^{sec} / PBF , if PBF < 1.$$

6. The method of claim 5, further comprising comparing
$$\{ {Z}_{i , j}^{{first}^{*}} \} to \{ {Z}_{i , j}^{{sec}^{*}} \}$$
to diagnose the possibility of disease.

7. The method of claim 1, wherein the step of obtaining a prebalancing factor includes obtaining sets of normalizing factors
$$\{ {r}_{i , j}^{first} \} and \{ {r}_{i , j}^{sec} \}$$
from the population group to account for variability within the first and second body parts.

8. The method of claim 7, wherein the step of obtaining a prebalancing factor further includes
obtaining a set of impedances
$$\{ {Z}_{i , j}^{first} \}$$
from the first body part and a set of impedances
$$\{ {Z}_{i , j}^{sec} \}$$
from the second body part;
utilizing
$$\{ {Z}_{i , j}^{first} \} and \{ {r}_{i , j}^{first} \}$$
to calculate a set of normalized impedances
$$\{ {Znorm}_{i , j}^{first} \} , and \{ {Z}_{i , j}^{sec} \} and \{ {r}_{i , j}^{sec} \}$$
to calculate a set of normalized impedances
$$\{ {Znorm}_{i , j}^{sec} \} ; and$$
averaging a subset of
$$\{ {Znorm}_{i , j}^{first} \}$$
and a subset of
$$\{ {Znorm}_{i , j}^{sec} \}$$
to obtain the prebalancing factor, the subsets formed by omitting normalized impedances that could correspond to anomalous electrical pathways.

9. The method of claim 8, wherein the step of obtaining the set of normalizing factors
$$\{ {r}_{i , j}^{first} \}$$
includes
applying nₑ voltage measurement electrodes to the first body part of a first member of the population group containing N_{g} members, where nₑ and N_{g} are integers greater than one;
measuring in the first member a set of voltages
$$\{ {V}_{i , j}^{first1} \} ,$$
where
$${V}_{i , j}^{first1}$$
is the voltage between an i^{th} voltage measurement electrode and a j^{th} voltage measurement electrode, the i^{th} and j^{th} voltage measurement electrodes chosen from among the nₑ voltage measurement electrodes; and
obtaining a reference specific impedance,
$${M}_{ref}^{first} ,$$
associated with a pair of reference electrodes chosen from among the nₑ voltage measurement electrodes.

10. The method of claim 9, wherein the step of obtaining the set of normalizing factors
$$\{ {r}_{i , j}^{first} \}$$
further includes
calculating a set of impedances
$$\{ {Z}_{i , j}^{first 1} \}$$
obtained from
$$\{ {V}_{i , j}^{first 1} \} ;$$
calculating a set of specific impedances
$$\{ {M}_{i , j}^{first 1} \}$$
where
$${M}_{i , j}^{first 1} = {V}_{i , j}^{first 1} / {d}_{i , j}^{first} and {d}_{i , j}^{first}$$
is a distance related to the distance between the i^{th} and j^{th} voltage measurement electrodes;
calculating a set of quotients
$$\{ {q}_{i , j}^{first 1} \}$$
where
$${q}_{i , j}^{first 1} = {M}_{i , j}^{first 1} / {M}_{ref}^{first} ;$$
and
calculating quotients for other members of the population group to obtain all quotients,
$$\{ {q}_{i , j}^{first K} \}$$
where K runs from one to N_{g}.

11. The method of claim 10, wherein the step of obtaining the set of normalizing factors
$$\{ {r}_{i , j}^{first} \}$$
further includes calculating the set according to
$${r}_{i , j}^{first} = \frac{1}{{N}_{g}} {\sum }_{K = 1}^{{N}_{g}} {q}_{i , j}^{first K} .$$

12. The method of claim 8, wherein the step of obtaining the set of impedances
$$\{ {Z}_{i , j}^{first} \}$$
includes
applying a plurality of current injection electrodes on the first body part; and
applying a plurality of voltage measurement electrodes on the first body part.

13. The method of claim 12, wherein the step of obtaining the set of impedances
$$\{ {Z}_{i , j}^{first} \}$$
includes
injecting a first current between a first current injection electrode and a second current injection electrode;
measuring a resultant voltage difference between a first voltage measurement electrode and a second voltage measurement electrode;
obtaining an impedance
$$\{ {Z}_{1 , 2}^{first} \}$$
from the resultant voltage difference between the first voltage measurement electrode and the second voltage measurement electrode; and
repeating the above steps with other electrodes to obtain the set of impedances
$$\{ {Z}_{i , j}^{first} \} .$$

14. The method of claim 13, wherein the step of obtaining a set of normalizing factors from the population group to account for variability within the first and second body parts includes obtaining a normalizing factor
$${r}_{i , j}^{first}$$
for each
$${Z}_{i , j}^{first} .$$

15. The method of claim 14, wherein the step of utilizing
$$\{ {Z}_{i , j}^{first} \} and \{ {r}_{i , j}^{first} \}$$
includes calculating a set of normalized impedances
$$\{ {Znorm}_{i , j}^{first} \}$$
according to
$${Znorm}_{i , j}^{first} = {Z}_{i , j}^{first} / {r}_{i , j}^{first} .$$

16. The method of claim 1, further comprising utilizing the electrical property after prebalancing to diagnose the possibility of disease in one of the first body part and the second body part

17. A system for prebalancing an electrical property obtained from at least one of a first body part and a second substantially similar body, the system comprising:
a prebalancing factor module for obtaining a prebalancing factor (PBF) from a population group to account for variability between the first body part and the second body part;
an electrode array for measuring an electrical property of at least one of the first body part and the second body part; and
a prebalancing module for utilizing the prebalancing factor to prebalance the electrical property.

18. The system of claim 17, wherein the first and second body parts are breasts.

19. The system of claim 17, wherein the electrode array includes a plurality of current injection electrodes and a plurality of voltage measurement electrodes.

20. The system of claim 19, wherein the electrical property is electrical impedance, and wherein
the plurality of current injection electrodes are used to inject currents into the first and second body parts; and
the plurality of voltage measurement electrodes are used to measure a set of impedances
$$\{ {Z}_{i , j}^{first} \} and \{ {Z}_{i , j}^{sec} \} .$$

21. The system of claim 20, wherein the prebalancing factor module prebalances
$$\{ {Z}_{i , j}^{first} \} and \{ {Z}_{i , j}^{sec} \}$$
to yield the sets
$$\{ {Z}_{i , j}^{{first}^{*}} \} and \{ {Z}_{i , j}^{{sec}^{*}} \} ,$$
where
$$\begin{matrix}{Z}_{i , j}^{{first}^{*}} = PBF \times {Z}_{i , j}^{first} and {Z}_{i , j}^{{sec}^{*}} = {Z}_{i , j}^{sec} , if PBF \geq 1 , and \\ {Z}_{i , j}^{{first}^{*}} = {Z}_{i , j}^{first} and {Z}_{i , j}^{{sec}^{*}} = {Z}_{i , j}^{sec} / PBF , if PBF < 1.\end{matrix}$$

22. The system of claim 21, further comprising a diagnosis module for comparing
$$\{ {Z}_{i , j}^{{first}^{*}} \} to \{ {Z}_{i , j}^{{sec}^{*}} \}$$
to diagnose the possibility of disease.

23. The system of claim 17, further comprising a normalizing factor calculation module for obtaining sets of normalizing factors
$$\{ {r}_{i , j}^{first} \} and \{ {r}_{i , j}^{sec} \}$$
to account for variability within the first and second body parts.

24. The system of claim 23, wherein the electrode array is used to obtain a set of impedances
$$\{ {Z}_{i , j}^{first} \}$$
from the first body part and a set of impedances
$$\{ {Z}_{i , j}^{sec} \}$$
from the second body part, which, together with the sets
$$\{ {r}_{i , j}^{first} \}$$
and
$$\{ {r}_{i , j}^{sec} \} ,$$
yield a set of normalized impedances
$$\{ {Znorm}_{i , j}^{first} \}$$
for the first body part, and a set of normalized impedances
$$\{ {Znorm}_{i , j}^{sec} \}$$
for the second body part, the system further comprising a prebalancing calculator module for obtaining the prebalancing factor after averaging of a subset of
$$\{ {Znorm}_{i , j}^{first} \}$$
and a subset of
$$\{ {Znorm}_{i , j}^{sec} \} ,$$
the subsets formed by omitting normalized impedances that could correspond to anomalous electrical pathways.

25. The system of claim 24, further comprising ne voltage measurement electrodes applied to the first body part of a first member of the population group containing N_{g} members, where nₑ and N_{g} are integers greater than one, to obtain a set of voltages
$$\{ {V}_{i , j}^{first1} \} ,$$
where
$${V}_{i , j}^{first 1}$$
is the voltage between an i^{th} voltage measurement electrode and a j^{th} voltage measurement electrode, the i^{th} and j^{th} voltage measurement electrodes chosen from among the nₑ voltage measurement electrodes.

26. The system of claim 25, further comprising a specific impedance calculation module to calculate a set of specific impedances
$$\{ {M}_{i , j}^{first 1} \}$$
from
$$\{ {V}_{i , j}^{first 1} \}$$
and to calculate a specific reference impedance
$${M}_{ref}^{first}$$
associated with a pair of reference electrodes chosen from among the nₑ voltage measurement electrodes, wherein
$$\{ {M}_{i , j}^{first 1} \} and {M}_{ref}^{first}$$
are used to calculate a set of normalizing quotient
$$\{ {q}_{i , j}^{first 1} \}$$
according to
$${q}_{i , j}^{first 1} = {M}_{i , j}^{first 1} / {M}_{ref}^{first} ,$$
and wherein other normalizing quotients for other members of the population group are calculated to obtain all quotients,
$$\{ {q}_{i , j}^{first K} \}$$
where K runs from one to N_{g}.

27. The system of claim 26, wherein the normalizing factor calculation module calculates a set of normalizing factors
$$\{ {r}_{i , j}^{first} \}$$
according to
$${r}_{i , j}^{first} = \frac{1}{{N}_{g}} {\sum }_{K = 1}^{{N}_{g}} {q}_{i , j}^{first K} .$$

28. The system of claim 27, further comprising a diagnosis module for utilizing the electrical property after prebalancing to diagnose disease.
